(19) United States
(12) Patent Application Publication
(10) Pub. No.: US 2009/0264671 A1
(43) Pub. Date: Oct. 22, 2009

(54) Method for Producing Biodiesel Using Supercritical Alcohols

(76) Inventors: Min Jeong Noh, Dejeon (KR); Ki Pung Yoo, Seoul (KR); Young Hae Choi, Seoul (KR)

Correspondence Address: MEYERTONS, HOOD, KIVLIN, KOWERT & GOETZEL, P.C., P.O. BOX 398, AUSTIN, TX, 78767-0398, US

(21) Appl. No.: 12/085,245

(22) PCT Filed: Nov. 16, 2006
(86) PCT No.: PCT/KR2006/004828

§ 371 Date: May 19, 2008

(30) Foreign Application Priority Data
Nov. 18, 2005 (KR) 10-2005-0110551

(51) Int. Cl. C07C 69/003

(52) U.S. Cl. 560/129

(57) ABSTRACT

Disclosed herein is a method for producing biodiesel in the form of fatty acid alkyl ester by esterifying oils-and-fats, including animal or vegetable oils-and-fats or waste thereof, with supercritical alcohol. According to the disclosed method, it is possible to produce high-purity fatty acid alkyl ester at low cost and high productivity.

### TECHNICAL FIELD

The present invention relates to the production of biodiesel, and more particularly to a method for producing biodiesel by esterifying animal or vegetable oils-and-fats or waste cooking oils containing these oils-and-fats, as raw materials, with alcohols, including methanol and the like, in conditions where the alcohols are maintained at a supercritical state, as well as a system for carrying out the production of biodiesel.

### BACKGROUND ART

Since the 20^{th} century, the production of petroleum among fossil fuels has enormously increased as the industry has developed, and petroleum has received attention as an energy source for industrial machines and transportations. However, petroleum resources have limited resources, and as confirmed in the two oil shocks of the 1970s, there is an urgent need to develop a new alternative energy source, due to various problems caused by a change in crude oil prices and the use of resources as weapons (OPEC, etc.).

Diesel engines are engines that use, as an energy source, diesel oil refined from crude oil, and are widely used in advanced countries due to low cost and excellent efficiency. However, in comparison with other fuels, diesel oil has problems in that it causes air pollution after combustion.

To solve such problems, diverse studies on alternative energy sources, which have physical properties similar to those of diesel oil and do not cause air pollution, have been conducted. For example, studies on fatty acid alkyl ester (hereinafter, referred to as “biodiesel”), which has physical properties similar to those of diesel oil and contributes to a reduction in air pollution, have been conducted.

Biodiesel oil is esterified oil, which is produced by allowing oils and fats, such as vegetable oils, animal fats or recyclable waste cooking oil, to react with alcohols in the presence of an acidic catalyst or alkaline catalyst.

Generally, biodiesel is produced by allowing alcohol to react with oil or fat in the presence of a heterogeneous catalyst of a strong base such as sodium hydroxide, or a strong acid such as sulfuric acid.

Prior methods of producing biodiesel using a strong acid catalyst include a method comprising allowing methyl acetate to react with butyl alcohol in the presence of a concentrated sulfuric acid catalyst (German Patent No. 1,909,434), and a Harrington's method comprising mixing sunflower oil with methanol at a molar ratio of 1:100 or more and allowing the mixture to react for 3-4 hours in the presence of a concentrated sulfuric acid catalyst (Harrington, Ind. Eng. Chem. Prod. Res. Dev., 24, pp 314-318, 1985). According to the Harrington's method, fatty acid methyl ester can be obtained with a purity of 40.7%.

Also, known techniques that use strong base catalysts include a technique suggested by B. Freedman, J.A.O.C.S, 61(10):1638-1643, and European Patent No. 301,643. These techniques disclose methods of preparing esters using a hydrophilic, strong base catalyst such as KOH, K₂₂ CO₃ or NaOH, and particularly, a method of producing biodiesel using a base catalyst in combination with an acid catalyst is commercially widely used.

With respect to the methods of producing biodiesel using catalysts, there has been a continued effort to increase catalytic reactivity in continuous processes (see Austria Patent No. PJ1105/88 (1988), French Patent No. 1,583,583, U.S. Pat. No. 3,852,315, etc.). Particularly, WO 91/05034, EP 409 177 and DE 3925514, owned by Henkel Corp, disclose methods for improving processing. Also, Korean Patents relating to the production of biodiesel by the use of catalysts include Korean Patent Laid-Open Publication Nos. 1999-024529, 1999-024530, 2003-0049614, 2003-0066246, 2004-0092930, 2005-0006032, 10-2004-0054318, and 10-2004-0084515, each of which mainly discloses a method of producing biodiesel using a catalyst. Meanwhile, in the production of biodiesel through a catalytic reaction, there is a problem in that free fat acid causes saponification with a catalyst (Wright, A report on ester interchange, Oil Soap, 21, 145-148 (1944)) to reduce the yield of biodiesel. Thus, methods for solving this problem were applied for patent protection (see Korean Patent Laid-Open Publication No. 10-2004-0087625).

The above-described prior techniques relate to producing biodiesel from animal and vegetable oils and fats and waste cooking oil using catalysts and have the following problems.

First, biodiesel is used in internal combustion engines such as automobile diesel engines, and thus, when biodiesel contains catalyst residue, it can causes problems such as engine corrosion and nozzle plugging.

Second, when fat and oil used as raw materials contain free fatty acid, saponification with a catalyst can occur, and thus the free fatty acid should be removed through pretreatment. Alternatively, soap components should be removed by washing the product with water after the production of biodiesel, and water used in the washing process should be suitably treated because it is disposed of as wastewater. Thus, when waste cooking oil having a high free fatty acid content is used, it makes the application of causes pretreatment and post-treatment processes necessary, resulting in a reduction in the economic efficiency of biodiesel production.

One of techniques, which have been recently widely investigated in advanced countries and research institutes to overcome the above-described problems, is transesterification with supercritical alcohol. Supercritical alcohol performs the esterification of oils even in the absence of a catalyst, and it was reported that, when supercritical alcohol contains free fatty acid, it can be methyl-esterified to produce biodiesel. Hideki Fukuda reported the synthesis of biodiesel through the use of supercritical alcohol (Hideki Fukuda, J. of Bioscience and Bioengineering Vol. 92. No. 5, pp 405-416, 2001), and Ayhan Demirbas reported studies on the synthesis of biodiesel through the use of supercritical alcohol (Ayhan Demirbas; Energy Conversion and Management, 44, pp 2093-2109, 2003). The reports of Yuichiro Warabi (Bioresource Technology, 91, pp 283-287, 2004) and Dada Kusdiana (Bioresource Technology 91, pp 289-295, 2004) showed that biodiesel could be produced using various kinds of supercritical alcohols, and the use of supercritical alcohol substantially eliminated the effects of free fatty acid and water.

Patents relating to the production of biodiesel by the use of supercritical alcohol include JP 2000-109883, JP 2001-524553, and U.S. Pat. No. 6,884,990 B2, U.S. Pat. No. 6,887,283 B1, US 2005/0033071 A1, and WO 2004/108873 A1. These patent documents include disclosures similar to those of the above-described papers, and solutions to increase reaction efficiency, but are disadvantageous in commercial terms, because these patents show limitations in terms of production cost and the like.

In the case of the above-described papers and patent documents, the desired purity of biodiesel could be produced either by using an excess amount of alcohol, considering that esterification is a reverse reaction as shown in Reaction Scheme 1, or by rapidly cooling the reaction product in order to prevent the reverse reaction:

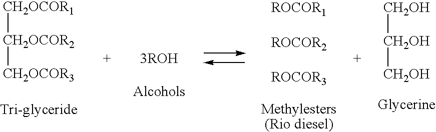

However, in view of the operating condition of supercritical alcohol, rapid cooling from a temperature of more than 300° C. can show a problem in terms of energy efficiency, and when a heat exchanger is used to overcome this problem, the reverse reaction between glycerin and methyl ester, as shown in Reaction Scheme 1 above, occurs, making it difficult to produce the desired purity of biodiesel. Also, when oil or fat is esterified at high temperatures, there are problems in that the thermal denaturation and carbonization of biodiesel occur, and such changes in physical properties and purity influence the quality of biodiesel for general use in diesel engines.

These days, the purity of fatty acid alkyl ester (FAME) as biodiesel for use as automobile fuel must satisfy a purity of 96.5% according to standards in Korea, USA, European and the like. However, the methods provided in said papers and patents cannot produce the desired purity of biodiesel due to a reverse reaction occurring during heat exchange.

### DISCLOSURE OF INVENTION

### Technical Problem

It is an object of the present invention to produce biodiesel by esterifying animal or vegetable oils and fats with supercritical alcohol in a high-temperature and high-pressure reactor in the absence of a catalyst and to produce eco-friendly, pure biodiesel by eliminating the problem of catalyst residue, which occurs in the prior biodiesel process that uses a catalyst, and eliminating a washing process for removing impurities resulting from the catalyst residue.

Another object of the present invention is to produce biodiesel regardless of the content of free fatty acid in oil as a raw material and to use waste cooking oil having a high free fatty acid content, directly as a raw material, because saponification occurring in the prior production method that uses a catalyst does not occur in the present invention, which adopts esterification without using any catalyst.

To achieve the above objects, the present invention provides a method for producing high-purity fatty acid alkyl ester using a single-stage or multi-stage reactor and a heat exchanger for minimizing the use of energy, and provides an optimized method for producing high-purity biodiesel in an economic manner depending on raw materials and operating conditions by solving problems associated with an irreversible reaction in esterification.

The present invention relates to a method for producing a high purity (a fatty acid alkyl ester content of more than 96.5%) of biodiesel in a continuous process. In the present invention, the use of energy is minimized by providing a high-pressure heat exchange that heats raw materials with a reactor temperature necessary for esterification, considering temperature and pressure conditions for making a supercritical alcohol phase. Also, the desired purity of biodiesel is produced by preventing a reduction in purity and yield caused by a reverse reaction occurring upon heat exchange, using a first-stage reactor and a purification column. When a raw material, from which the desired purity of biodiesel can not be produced using said method, is used, glycerin is removed from a reaction product generated in a first-order reaction, and the raw material (fat or oil) from which glycerin has been removed is produced into the desired purity of biodiesel in a second-stage reactor, thus producing biodiesel with a yield of 97.7%.

As a result, a main object of the present invention to provide a method for producing fatty acid alkyl ester (biodiesel) and other byproducts from oil, fat or waste cooking oil using supercritical alcohol, such that the produced biodiesel satisfies standards. In the method, a heat exchanger is provided to minimize energy required for producing biodiesel in a continuous process, and a reduction in purity caused by a reverse reaction resulting from the use of the heat exchanger is prevented through the use of one or two or more supercritical reactors, thus producing the desired purity of biodiesel. Also, biodiesel can be produced from each of various raw materials.

### Technical Solution

According to one embodiment of the present invention, there is provided a method for producing biodiesel in the form of fatty acid alkyl ester by esterifying oils-and-fats, including vegetable or animal oils-and-fats or wastes thereof, the method comprising the steps of:

(a) pumping alcohol and oils-and-fats under pressure into a mixer, in which they are uniformly mixed with each other;

(b) elevating the temperature of the mixture through a heat exchanger;

(c) heating the temperature-elevated mixture to a predetermined temperature;

(d) subjecting the mixture to esterification in conditions where the alcohol is maintained at a supercritical state;

(e) heat-exchanging the esterification product with a subsequent mixture of alcohol and oil-and-fat in the heat exchanger;

(f) reducing the pressure of the heat-exchanged product to separate and recover the alcohol; and

(g) separating and recovering fatty acid alkyl ester from the reaction product from which the alcohol has been removed.

According to the present invention, when the content of the recovered fatty acid alkyl ester does not reach the desired level, said supercritical esterification step is additionally conducted more than one time.

According to still another embodiment of the present invention, the inventive method further comprises, before the step of pumping the alcohol into the mixer, the step of maintaining the alcohol above the critical points thereof through a separate heat exchanger.

According to yet still another embodiment of the present invention, the method further comprises the step of removing dissolved oxygen present in each of the oils-and-fats and alcohol used as raw materials.

Hereinafter, the present invention will now be described in further detail with reference to the accompanying drawings, which show one example of a preferred system for carrying out the inventive method.

Oils and fats, which are used as raw materials in the inventive method, are selected from the group consisting of vegetable and animal oils and fats, and wastes thereof, and specific examples thereof include soybean oil, rapeseed oil, sunflower seed oil, corn oil and palm oil. Also, alcohol is preferably selected from among alcohols having 1-8 carbon atoms, and mixtures of two or more thereof.

The system illustrated in FIG. 1 broadly consists of four sections: a raw material storage and supply section indicated by reference numerals 100s, a first reactor and separation section indicated by reference numerals 200s, a second reactor and separation section indicated by reference numerals 300s, and a purification and storage section indicated by reference numerals 400s.

Before raw material alcohol stored in a storage tank 101 is pressurized by a pressurizing pump 103, oxygen contained in the alcohol is preferably removed in a dissolved oxygen-removing unit 102. Meanwhile, before oils-and-fats stored in a storage tank 104 are pressurized to the desired pressure by a pressurizing pump 106, oxygen contained in a dissolved oxygen-removing unit 105 is preferably removed. When oxygen dissolved in said materials is removed before esterification, a high quality of biodiesel can be obtained. Herein, the removal of oxygen from the raw materials can be performed by heating the raw materials, treating the raw materials in a vacuum, or injecting inert gas, for example, nitrogen or helium gas, into the raw materials.

The raw materials are pressurized by the respective pressurizing pumps 103 and 106 to the desired pressure, so that they are supplied into a mixer 107, in which they are uniformly mixed with each other. When the raw materials are uniformly mixed using the mixer 107 as described above, the reaction therebetween will efficiently occur to increase yield. The mixing for increasing the blending of oils-and-fats with alcohol can be achieved using a mixer that uses mechanical force in the inside or outside thereof. Also, to order for oils-and-fats to be well mixed with alcohol, it is effective to make alcohol supercritical before it is supplied into the mixer 107. FIG. 2 illustrates the construction of a heat exchange for making alcohol supercritical. As illustrated in FIG. 2, an alcohol heat exchanger 208 can be provided in the rear of the alcohol-pressurizing pump 103, such that alcohol can be transferred into the mixer while it is maintained at a supercritical state. When alcohol becomes a supercritical state as described above, the effect of uniformly mixing alcohol can be maximized due to the characteristics of the supercritical fluid. Herein, the heat exchanger has substantially the same construction as that of a first heat exchanger 201.

The alcohol and oils-and-fats, which have been uniformly mixed with each other in the mixer 107, are heated through a first heat exchanger 201, and are heated to the desired temperature in a first heating furnace 202. Because the mixture of alcohol and oils-and-fats is pre-heated through the first heat exchanger 201, an energy source for the first heating furnace 202 requires energy corresponding to additional energy resulting from the ability of the first heat exchanger 201 and the heat loss of other units. Furthermore, in the case of supercritical esterification, an exothermic reaction progresses in a first reactor 203, and heat resulting from the exothermic reaction is exchanged with the mixture of alcohol and oils-and-fats in the first heat exchanger 201, and thus energy required in the first heating furnace 202 is very small.

The mixture of alcohol and oils-and-fats, heated in the first heating furnace 202, is esterified in the first reactor 203 under conditions where alcohol becomes supercritical. The first reactor 203 can be a tube type or autoclave type and is designed considering residence time, etc. To maintain alcohol at a supercritical state in the first reactor 203, the temperature of the reactor is set to the critical temperature or higher of the alcohol, preferably 300-400° C., and more preferably 350-400° C., and the pressure of the reactor is set to the critical pressure or higher of the alcohol, preferably 10-20 MPa, and more preferably 10-12 MPa. The residence time in the reactor is 1 minute or more, preferably 5-60 minutes, and more preferably 10-20 minutes. Also, the volume ratio of alcohol to oils-and-fats in the reactor is 0.5-10:1, and preferably 0.5-2:1.

The first heat exchanger 201 serves to elevate the temperature of fluid flowing from the mixer 107 using the temperature of fluid flowing out from the first reactor 203, i.e., the temperature of the esterification product, and is operated at high pressure. Thus, a heat exchanger capable of maintaining high pressure should be used as the first heat exchanger.

The esterification product from the first heat exchanger 201 is adjusted from high pressure to atmospheric pressure or low pressure by means of a first pressure-reducing valve 204 and is introduced into a first alcohol recovery unit 205. In the first alcohol recovery unit 205, biodiesel produced in the first reactor 203 is subjected to a process in which alcohol is recovered for use as a raw material in order to overcome a reduction in purity caused by a reverse reaction occurring in the first heat exchanger 201 required for heat recovery and to remove glycerin. Herein, alcohol is discharged through the top of the first alcohol recovery unit 205, and the discharged alcohol is passed through an alcohol condenser 207 to have the desired temperature and is transferred into the alcohol storage tank 110. The transferred alcohol is recycled as a raw material without any additional treatment. Meanwhile, biodiesel, unreacted oil and fat, and glycerin, are discharged through the bottom of the first alcohol recovery unit 205, and these compounds are subjected to phase separation in a tank for first biodiesel/oil-and-fat/glycerin separator. In the three component separator 206, the biodiesel and the oil-and-fat are present as a single phase in the upper layer, and glycerin is present as a single phase in the lower layer. The phase separation will occur only when alcohol is present at a concentration of less than 1%, and if complete phase separation is not achieved, the yield and purity of biodiesel cannot satisfy standards due to the incorporation of glycerin.

When biodiesel among fluid recovered from the upper layer of the first separator 206 has the desired purity, preferably a purity of more than 93.5%, it can be sent directly to a biodiesel storage tank 401 without being passed through a second reactor/separator.

Before biodiesel is stored in the storage tank 401, the purity thereof can be increased using, for example, a purification column unit 402. The purification column unit 402 can also consist of a plurality of purification columns arranged in series or in parallel. A material filled in the purification preferably has a property of non-absorbing fatty acid alkyl ester, and typical examples of this filler material include activated carbon, silica gel, ion exchange resin, diatomite, bentonite, pearlite, and mixtures of two or more thereof.

The production of biodiesel through the above-described first-order reaction can be used when the purity of fatty acid alkyl ester can be increased by about 3% in the purification column unit 402. Also, it can be used when the purity of biodiesel discharged from the top of the first separator 206 is the desired purity or more, and preferably 93.5% or more.

Glycerin recovered from the bottom of the first separator 206 is passed through a glycerin purification unit depending on the desired standard and is transferred into a glycerin storage unit 403.

Meanwhile, when the purity of biodiesel transferred from the top of the first separator 206 does not reach the desired purity, it is preferable to subject the biodiesel to a second-order reaction, because it is difficult to adjust the biodiesel to the desired purity only with the purification column unit 402. The second-order reaction is conducted in substantially the same manner as the first-order reaction, and will be briefly described.

Fluid flowing from the first separator 206 is pressurized by a second biodiesel/oil pressurizing pump 301, and alcohol A passed through the alcohol storage tank 101 and the dissolved oxygen-removing unit 102 is pressurized by a second alcohol pressurizing pump 302. The pressurized substances are uniformly mixed in a second mixer 303, and the mixture is transferred into a second heat exchanger 304. The transferred fluid is heated in the same manner as in the first heat exchanger 201, and is heated in a second heating furnace 305 to the desired temperature. The heated fluid is subjected to a final reaction in a second-stage reactor 306, and the reaction product is passed through the second heat exchanger 304 to recover energy, is adjusted to atmospheric pressure or low pressure through a second pressure-reducing valve 307 and transferred into a second alcohol recovery unit 308. The operating principle of the second alcohol recovery unit 308 is the same as the first alcohol recovery unit 205. The alcohol discharged through the top of the recovery unit 308 is transferred into an alcohol condenser 207, and it is then transferred into the alcohol storage tank 101 and recycled as a raw material. Through the bottom of the second alcohol recovery unit 308, biodiesel and glycerin are transferred into a second biodiesel/glycerin separator 309. Biodiesel recovered from the top of the second separator 309 is stored in a biodiesel storage tank 401. Glycerin recovered from the bottom of the secondary separator 309 is passed through a glycerin purification unit 404 and then stored in a glycerin storage tank 403.

Biodiesel subjected to the second-order reaction can also be purified through a purification column unit 402, if necessary. The conditions of the second-order reaction can be the same as or different from those of the first-order reaction.

### ADVANTAGEOUS EFFECTS

According to the present invention, it is possible to produce high-purity fatty acid alkyl ester in continuous reactors at low cost and high productivity without using any catalyst by subjecting animal or vegetable fats-and-oils and alcohols having various carbon numbers to either a combination of first-order reaction and column purification or the first-order reaction, recovering energy from the first-order reaction product and removing glycerin to eliminate the cause of a reverse reaction, and then subjecting the first-order reaction product to a second-order reaction.

### BRIEF DESCRIPTION OF THE DRAWINGS

FIG. 1 is a schematic diagram showing a system for producing biodiesel according to one embodiment of the present invention.

FIG. 2 is a schematic diagram showing the construction of a heat exchanger for making alcohol supercritical before supplying the alcohol into a mixer, according to another embodiment of the present invention.

### BEST MODE FOR CARRYING OUT THE INVENTION

Hereinafter, the present invention will be described in further detail with reference to examples. However, it will be obvious to those skilled in the art that these examples are for illustrative purposes only and the scope of the present invention is not limited thereto.

### Mode for the Invention

### Example 1

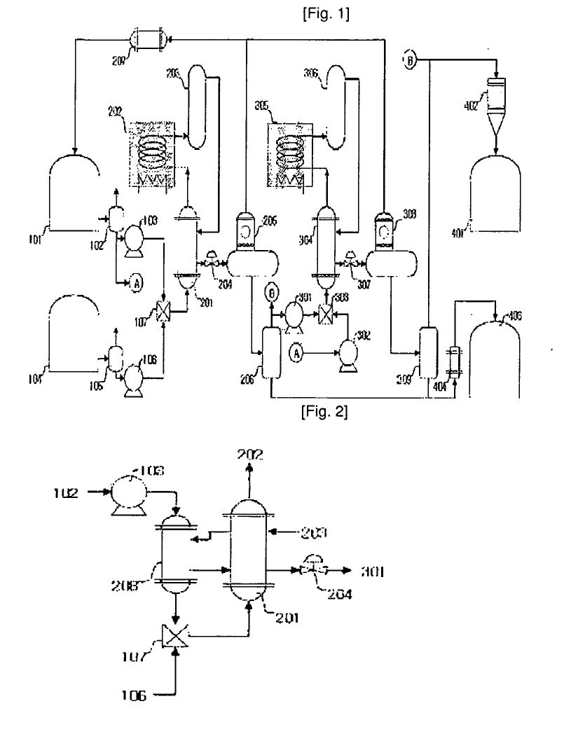

Biodiesel was continuously produced in a system designed as shown in FIG. 1. A reactor used in the production was a tubular reactor.

After dissolved oxygen was removed from oils-and-fats and alcohols as raw materials, the raw materials were pumped under pressure into a mixer, in which they were mixed with each other. The mixture was preheated to a predetermined temperature through a heat exchanger and a heating furnace, and maintained at the desired temperature in a reactor. Then, the reaction product was cooled in a cooler, the pressure thereof was reduced by means of a pressure-reducing valve, and a sample was collected from the product.

Herein, the pressure for pumping the raw materials was 80-200 MPa, the preheated temperature was 80-250° C., and the temperature of the reactor was 250-400° C. Also, the reactor was a tubular reactor, and the residence time in the tubular reactor was 5-60 minutes.

In the production of biodiesel, the flow rate of the raw materials were controlled in volume by the respective high-pressure metering pump, and the degree of a quantitative reaction was examined by comparing the amounts collected from the introduced raw materials.

The reaction product was a fluid mixture of biodiesel, glycerin, oil-and-fat, and alcohol. Excessive alcohol was removed using a vacuum evaporator, and the remaining fluid was left to stand in a separation funnel, so that it was phase-separated into biodiesel and oil in the upper layer and glycerin in the lower layer. After the glycerin in the lower layer was removed, the fatty acid alkyl ester in the upper layer was analyzed to measure the purity thereof. The measurement of purity was performed according to the method of EN 14103 and KS M 2413-2004.

The oils-and-fats used in the above experiment were soybean oil, corn oil, palm oil, rapeseed oil, and rice bran oil-waste cooking oil, which are commercially available in Korea. Among the raw materials, soybean oil consisted of soybean oil extracted with supercritical carbon dioxide, and soybean oil extracted with hexane. Also, the rice bran oil-waste cooking used was obtained by extracting waste cooking oil generated in the frying of chicken with hexane and collecting components dissolved in the hexane.

Alcohols used in the above experiment were methanol, ethanol, 1-propanol, 1-butanol and 1-octanol. Considering the critical conditions of each of the alcohols, shown in Table 1, operating conditions for the production of biodiesel were determined.

TABLE 1

| | Critical | Operating | Critical | |
|---|---|---|---|---|
| Alcohols | temperature | temperature | pressure | Operating pressure |
| Methanol | 239° C. | More than 249° C. | 8.09 MPa | More than 9.09 MPa |
| Ethanol | 243° C. | More than 253° C. | 6.38 MPa | More than 7.38 MPa |
| 1-propanol | 264° C. | More than 274° C. | 5.06 MPa | More than 6.06 MPa |
| 1-butanol | 287° C. | More than 297° C. | 4.90 MPa | More than 5.90 MPa |
| 1-octanol | 385° C. | More than 395° C. | 2.86 MPa | More than 3.86 MPa |

Meanwhile, when the biodiesel obtained in the first-order reaction did not reach a purity of 96.5%, it was subjected to a second-order or third-order reaction in the same manner as in the first-order reaction, so that a biodiesel having a fatty acid alkyl ester content of 97.7% could be produced.

Table 2 below shows the fatty acid alkyl ester content of each of the raw materials used in the first-order reaction. The results in Table 2 were obtained in the following conditions: a reactor temperature of 380° C., a reactor pressure of 10 MPa, and a reactor residence time of 10 minutes. Alcohol used in Examples shown in Table 2 was methanol, and the volume ratio of oil and fat to methanol was 1 (oil and fat): 2 (methanol).

TABLE 2

| | | | Fatty acid |
|---|---|---|---|
| Example | Oil used | | methyl ester content |
| 1 | ybean oil | Commercially available | 84% |
| 2 | | extracted with hexane | 86% |
| 3 | | extracted with super- | 90% |
| | | critical CO₂ | |

| | | |
|---|---|---|
| 4 | Corn oil | 82% |
| 5 | Rapeseed oil | 65% |
| 6 | Palm oil | 75% |
| 7 | Waste cooking oil (rice bran oil for | 88% |
| | chicken frying) | |

Table 3 below shows a change in the content of fatty acid alkyl ester with a change in the kind of alcohol. In Examples shown in Table 3, commercially available soybean oil was used as an oil raw material, the volume of oil to alcohol was 1:2, and the conditions of the reactor were as follows: a temperature of 380° C., a pressure of 10 MPa, and a residence time of 10 minutes. Also, the results in Table 3 were obtained in the first-order reaction.

TABLE 3

| Examples | Alcohols | Fatty acid alkyl ester contents |
|---|---|---|
| 8 | Methanol | 84% |
| 9 | Ethanol | 78% |
| 10 | 1-propanol | 52% |
| 11 | 1-butanol | 23% |
| 12 | 1-octanol | 5% |

Table 4 below shows a change in the content of fatty acid alkyl ester with a change in the reactor temperature. In Examples shown in Table 4, commercially available soybean oil and methanol were used, and the volume ratio of soybean oil to methanol was 1:2. Also, the pressure of the reactor was set to 10 MPa, and the residence time in the reactor was 10 minutes. As can be seen in Table 4, fatty acid alkyl ester was produced in the temperature range of 300-400° C. The results in Table 4 were obtained in the first-order reaction.

TABLE 4

| Examples | Temperatures | Fatty acid methyl ester contents |
|---|---|---|
| 13 | 300° C. | 25% |
| 14 | 325° C. | 52% |
| 15 | 350° C. | 78% |
| 16 | 375° C. | 82% |
| 17 | 400° C. | 75% |

In the results of Table 4, in the case of 400° C., the collected sample showed a dark brown color together a severe odor, and these phenomena did not appear at around 380° C.

Table 5 below shows a change in the content of fatty acid methyl ester with a change in pressure. In Examples shown in Table 5, commercially available soybean oil and methanol were used at a volume ratio of 1:2, the temperature of the reactor was set to 380° C., and the residence time in the reactor was 10 minutes. As can be seen from the results of Table 5, fatty acid alkyl ester was prepared in the pressure range of 10-20 MPa. Also, the results of Table 5 were obtained in the first-order reaction, and there was little or no change in the change of fatty acid methyl ester with a change in pressure.

TABLE 5

| Examples | Pressures | Fatty acid methyl ester contents |
|---|---|---|
| 18 | 10 MPa | 84% |
| 19 | 15 MPa | 83% |
| 20 | 20 MPa | 80% |

Table 6 below shows experiment results for a change in the content of fatty acid methyl ester with a change in the residence time in the reactor. In Examples shown in Table 6, commercially available soybean oil and methanol were used at a volume ratio of 1:2, and the temperature and pressure of the reactor were set to 380° C. and 10 MPa, respectively. The results in Table 6 were obtained in the first-order reaction. As can be seen from the experimental results in Table 6, when the residence time was insufficient as in Example 21, the content of fatty acid methyl ester was low, and when the residence time was increased as in Example 23, a reverse reaction could occur, resulting in a reduction in the fatty acid methyl ester content. Also, it was found that a reduction in flow rate, resulting from with an increase in the residence time as in Example 25, could lead to a reduction in reaction rate due to insufficient mixing in the tubular reactor. Thus, it could be seen that, when the residence time in the reactor is increased, sufficient mixing is required.

TABLE 6

| Examples | Residence time | Fatty acid methyl ester contents |
|---|---|---|
| 21 | 5 minutes | 57.4% |
| 22 | 10 minutes | 82.8% |
| 23 | 20 minutes | 72.7% |
| 24 | 30 minutes | 64.4% |
| 25 | 60 minutes | 48.1% |

Table 7 below shows experimental results for a change in the content of fatty acid alcohol ester with a change in the volume ratio of oil-and-fat to alcohol. In Examples shown in Table 7, commercially available soybean oil and methanol were used, the temperature and pressure of the reactor were set to 380° C. and 10 MPa, respectively, and the residence time in the reactor was 10 minutes. The results in Table 7 were obtained in the first-order reaction. As can be seen in Table 7, even when the amount of methanol used was increased, the content of fatty acid methyl ester was not greatly changed. This suggests that the amount of alcohol used can be reduced in actual processes.

TABLE 7

| | Soybean oil:methanol | |
|---|---|---|
| Examples | (Vol %:%) | Fatty acid methyl ester contents |
| 26 | 1.0:0.5 | 77.5% |
| 27 | 1.0:1.0 | 84.0% |
| 28 | 1.0:2.0 | 81.9% |
| 29 | 1.0:3.0 | 82.8% |

### Example 30

### Production of Biodiesel by Second-Order Reaction

Fatty acid methyl ester obtained according to the method of Example 1 was subjected to a second-order reaction according to the method described in Example 1, and the content of fatty acid methyl ester in the product was analyzed. The product obtained in the first-order reaction had a fatty acid methyl ester content of 78.7%, and the second-order reaction was carried out using the first-order reaction product and methanol at a volume ratio of 1:1 in the following conditions: a reactor temperature of 350° C., a reactor pressure of 10 MPa, and a reactor residence time of 13 minutes. The analysis results showed that the content of fatty acid methyl ester in the second-order reaction product was 97.7%, and the total glycerin content (wt %) in the product was 0.028%. Herein, the content of fatty acid methyl ester content was analyzed according to KS M 2413-2004, and the total glycerin content was analyzed according to KS M 2412-2004.

### Example 31

### Production of Biodiesel by Second-Order Reaction

A raw material having a fatty acid methyl ester of 81.3% was subjected to a second-order reaction according to the same method as in Example 30, thus obtaining a product having a fatty acid methyl ester of 97.2%. The analysis of the fatty acid methyl ester content was carried out according to KS M 2413-2004.

### Example 32

### Production of Biodiesel by Third-Step Reaction

The second-order reaction product obtained in Example 30 was subjected to a third-step reaction according to the same method as in Example 1. As a result, biodiesel having a fatty acid methyl ester of 98.4% was produced.

### Example 33

### Purification for Increasing Content of Biodiesel

The biodiesel obtained in Example 1 was subjected to a column purification experiment. 1 liter of a sample having a fatty acid methyl ester content of 72.7% was passed through 50 g of charcoal at 60° C., and the purity thereof was then measured. As a result, biodiesel having a fatty acid methyl ester content of 79.6% was produced. Also, biodiesel having a fatty acid methyl ester content of 94.7% was treated according to the above-described method, thus producing biodiesel having a fatty acid methyl ester content of 96.9%.

### Example 34

### Test of Biodiesel Performance

Biodiesel having a fatty acid methyl ester of 87%, produced according to the method of Example 1, was tested whether it satisfies domestic quality standards. A sample used in the test was prepared by mixing 80 vol % of diesel oil (purchased from an SK service station on May, 2004) with the biodiesel at a mixing ratio of 80 (diesel oil):20 (biodiesel), and the test results for quality standards are shown in Table 8.

TABLE 8

| | Quality | Test | |
|---|---|---|---|
| Test items | standards | results | Test methods |
| Fatty acid methyl ester content | 20.0 ± 3 | 17.1 | EN 14078 |
| (wt %) | | | |
| Pour point (° C.) | Less than 0.0 | −15.0 | KS M 2016-2005 |
| Flash (° C.) | More than 40 | 49 | KS M 2010-2004 |
| Kinematic viscosity (40° C., mm²/s) | 1.9-5.5 | 2.784 | KS M 2014-2004 |
| Distillation (distillation temperature | Less than 360 | 343.0 | ASTM D 86 |
| (° C.) at which 90% of fuel | | | |
| evaporates) | | | |
| Content (wt %) of carbon residue in | Less than 0.15 | 0.20 | KS M 2017-2001 |
| 10% residual oil | | | |
| Sulfur content (mg/kg) | Less than 430 | 166 | KS M 2027-2005 |
| Ash content (wt %) | Less than 0.02 | 0.001 | KS M ISO 6245-2003 |
| Cetane number (cetane index) | More than 45 | 51.8 | KS M 2610-2001 |
| Copper corrosion (100° C., 3 hr) | Less than 1 | 1 | KS M 2018-2002 |
| Filter plugging point (° C.) | Less than −16 | −5 | KS M 2411-2001 |
| Density at 15° C. (kg/m³) | 815-855 | 841.7 | KS M ISO 12185-2003 |
| Total acid number (mg KOH/g) | Less than 0.10 | 0.7 | KS M ISO 6618-2003 |
| Lubricity at 60° C. (HFRR wear scar | Less than 460 | 202 | KS M ISO 12156-1-2001 |
| diameter □) | | | |

### INDUSTRIAL APPLICABILITY

As apparent from the foregoing, according to the present invention, it is possible to produce high-purity fatty acid alkyl ester in continuous reactors at low cost and high productivity without using any catalyst by subjecting animal or vegetable fats-and-oils and alcohols having various carbon numbers to either a combination of first-order reaction and column purification or the first-order reaction, recovering energy from the first-order reaction product and removing glycerin to eliminate the cause of a reverse reaction, and then subjecting the first-order reaction product to a second-order reaction.

1. A method for producing biodiesel in the form of fatty acid alkyl ester by esterifying oils and-fats, including vegetable or animal oils and fats or wastes thereof, with alcohol, the method comprising the steps of:
(a) pumping alcohol and oils and fats under pressure into a mixer, in which they are uniformly mixed with each other;
(b) elevating the temperature of the mixture through a heat exchanger;
(c) heating the temperature-elevated mixture to a predetermined temperature;
(d) subjecting the mixture to esterification in conditions where the alcohol is maintained at a supercritical state;
(e) heat-exchanging the esterified product with a subsequent mixture of alcohol and oils-and-fats in the heat exchanger;
(f) reducing the pressure of the heat-exchanged product to separate and recover the alcohols; and
(g) separating and recovering fatty acid alkyl ester from the reaction product from which the alcohols have been removed.

2. The method of claim 1, wherein, when the content of the recovered fatty acid alkyl ester does not reach a predetermined level, said supercritical esterification step is additionally conducted more than one time.

3. The method of claim 1, which further comprises, before pumping the alcohol into the mixer, a step of maintaining the alcohol above the critical points thereof through a separate heat exchanger.

4. The method of claim 1, wherein the heat exchanger is a cross heat exchanger, or an exchanger of conducting heat exchange using an external heat medium.

5. The method of claim 4, wherein the heat exchanger uses the external heat medium, and comprises a heat exchanger network, which has a function of controlling esterification by increasing the amount of use of the heat medium.

6. The method of claim 1, wherein the oils-and-fats are selected from the group consisting of soybean oil, rapeseed oil, sunflower seed oil, corn oil, palm oil, and mixtures of two or more thereof.

7. The method of claim 1, wherein the alcohol is selected from the group consisting of alcohols having 1-8 carbon atoms, or mixtures of two or more thereof.

8. The method of claim 1, wherein the temperature of the supercritical esterification is maintained above the critical temperature of the alcohols.

9. The method of claim 1, wherein the temperature of the supercritical esterification is 300-400° C.

10. The method of claim 9, wherein the temperature of the supercritical esterification is 350-400° C.

11. The method of claim 1, wherein the pressure of the supercritical esterification is maintained above the critical pressure of the alcohols.

12. The method of claim 11, wherein the pressure of the supercritical esterification is 10-20 MPa.

13. The method of claim 11, wherein the pressure of the supercritical esterification is 10-12 MPa.

14. The method of claim 1, wherein the residence time of the reaction mixture in the supercritical esterification is more than 1 minute.

15. The method of claim 14, wherein the residence time is 5-50 minutes.

16. The method of claim 15, wherein the residence time is 10-20 minutes.

17. The method of claim 1, wherein the volume of the alcohols used in the esterification is 0.5-10 times the volume of the oils-and-fats.

18. The method of claim 17, wherein the volume of the alcohols used in the esterification is 0.5-2 times the volume of the oils-and-fats.

19. The method of claim 2, wherein the conditions of the first-order esterification are different from those of the second-order or higher-order esterification.

20. The method of claim 1, wherein the alcohol recovered in the step f) is recycled as a raw material.

21. The method of claim 1, which further comprises a step of purifying the recovered biodiesel.

22. The method of claim 1, which further comprises a step of removing dissolved oxygen present in each of the oils-and-fats and the alcohol.
